(19) United States
(12) Patent Application Publication
(10) Pub. No.: US 2021/0228098 A1
(43) Pub. Date: Jul. 29, 2021

(54) PULSE WAVE CONDUCTION PARAMETER MEASUREMENT SYSTEM AND METHOD

(71) Applicant: SHENZHEN DARMA TECHNOLOGY CO., LTD., Shenzhen City, Guangdong Provina (CN)

(72) Inventors: Shaochun ZHUANG, Shenzhen City (CN); Fei YE, Shenzhen City (CN); Renku CHEN, Shenzhen City (CN)

(73) Assignee: Shenzhen Darma Technology Co., Ltd., Shenzhen City, Guangdong Provina (CN)

(21) Appl. No.: 17/051,134

(22) PCT Filed: Apr. 28, 2018
(86) PCT No.: PCT/CN2018/085200

§ 371 Date: Oct. 27, 2020

(51) Int. Cl. A61B 5/021; A61B 5/00; G01H 9/00

(52) U.S. Cl. CPC A61B 5/02125; A61B 5/7239; G01H 9/004; A61B 5/6892; A61B 5/6822

(57) ABSTRACT

A pulse wave conduction parameter measurement system and method comprises: acquiring, by one or more processors, first vibration information of a supine subject from a first fiber optic sensor, the first fiber optic sensor being configured to be placed under a back region corresponding to the fourth thoracic vertebral body of the supine subject (step 711); acquiring, by the one or more processors, second vibration information of the supine subject from a second fiber optic sensor, the second fiber optic sensor being configured to be placed under a lumbar region corresponding to the fourth lumbar body of the supine subject (step 713); and generating, by the one or more processors, first hemodynamic related information on the basis of the first vibration information, and generating second hemodynamic related information on the basis of the second vibration information (step 715), thereby determining an aortic Pulse Wave Transit Time of the supine subject (step 719).

### FIELD OF THE INVENTION

The present invention relates generally to the field of a pulse wave conduction parameter measurement system and method, and particularly relates to a non-invasive pulse wave conduction parameter measurement system and method.

### BACKGROUND OF THE INVENTION

The description herein only provides background information related to the application, and do not necessarily constitute prior art.

Worldwide, cardiovascular and cerebrovascular diseases are an important cause of morbidity and death, and morbidity and death caused by cardiovascular and cerebrovascular diseases are related to arterial vascular diseases. For example, angina pectoris and myocardial infarction are related to coronary artery disease; stroke is related to cerebral artery disease, and intermittent claudication is related to lower extremity arterial disease. The two main types of arterial lesions include structural lesions and functional lesions. Structural lesions are manifested as vascular obstruction, such as atherosclerosis; and functional lesions are manifested as changes in vascular function, such as vascular sclerosis. While, the elasticity change of arterial wall is the cause of the occurrence and development of various cardiovascular events.

The cyclical contraction and relaxation of the heart can not only cause changes in the flow rate and flow of blood in arteries, but also generate pulse waves that propagate along the blood vessel wall. Pulse Wave Velocity (PWV) is related to the elasticity of arteries. Generally, the greater the stiffness of the blood vessel, the faster the pulse wave velocity. Therefore, the degree of arterial elasticity can be assessed by measuring the pulse wave velocity.

### SUMMARY OF THE INVENTION

### Technical Problem

The technical problem to be solved by the embodiment of the invention is to provide a non-invasive pulse wave conduction parameter measurement system and method for the technical problems related to the detection of central vascular diseases in the prior art.

### Technical Solutions to Problems

### Technical Solutions

In order to solve the technical problems, at an aspect, a method in accordance with one embodiment of the present invention comprises: acquiring first vibration information of a supine subject from a first fiber-optic sensor by one or more processors, the first fiber-optic sensor being placed under the back section corresponding to the fourth thoracic vertebra of a supine subject; acquiring second vibration information of the supine subject from a second fiber-optic sensor by one or more processors, the second fiber-optic sensor being placed under a lumbar section corresponding to the fourth lumbar vertebra of the supine subject; generating first hemodynamic related information on the basis of the first vibration information, and generating second hemodynamic related information on the basis of the second vibration information by one or more processors; determining an aortic valve opening time of the supine subject on the basis of the first hemodynamic related information, and determining an pulse wave arrival time of the supine subject on the basis of the second hemodynamic related information by one or more processors; and determining an aortic Pulse Wave Transit Time of the supine subject on basis of the aortic valve opening time and the pulse wave arrival time by one or more processors.

Preferably, the first fiber-optic sensor or the second fiber-optic sensor comprise: an optical fiber, disposed substantially in a plane; a light source, coupled with one end of one or more optical fibers; a receiver, coupled to the other end of one optical fiber, and configured to sense changes in the intensity of light transmitted through the optical fiber; and a mesh layer, composed of meshes with openings; the mesh layer is in contact with the surface of the optical fiber.

Preferably, the step of generating first hemodynamic related information on the basis of the first vibration information, and generating second hemodynamic related information on the basis of the second vibration information by one of more processors, further comprises step of: filtering and scaling the first vibration information and the second vibration information to generate the first hemodynamic related information and the second hemodynamic related information.

Preferably, the step of determining an aortic valve opening time of the supine subject on the basis of the first hemodynamic related information by one or more processors, further comprises steps of: performing a second-order differential calculation on the first hemodynamic related information; performing a feature search to a waveform of the first hemodynamic related information after the second-order differential calculation to determine the highest peak in a cardiac cycle; and determining the aortic valve opening time of the supine subject based on the highest peak.

Preferably, the method further comprises steps of: acquiring a distance between the first fiber-optic sensor and the second fiber-optic sensor in a body height direction to generate an aortic pulse wave conduction distance by one or more processors; and determining an aortic Pulse Wave Velocity on the basis of the aortic pulse wave conduction distance and the aortic Pulse Wave Transit Time.

Preferably, the method further comprising step of: sending at least one of the aortic Pulse Wave Transit Time and the aortic Pulse Wave Velocity to one or more output device, by the one or more processors.

At another aspect, a system provided in the present invention, comprises: a first fiber-optic sensor, being configured to be placed in an area corresponding to the fourth thoracic vertebra of a supine subject to acquire first vibration information of the supine subject; a second fiber-optic sensor, being configured to be placed in an area corresponding to the fourth lumbar vertebra of the supine subject to acquire second vibration information of the supine subject; one or more processors; and one or more computer-readable storage medium having instructions stored thereon, which when being executed by the one or more processor, cause the one or more processors to perform steps of: acquiring the first vibration information of the supine subject from the first fiber-optic sensor; acquiring the second vibration information of the supine subject from the second fiber-optic sensor; generating first hemodynamic related information on the basis of the first vibration information, and generating second hemodynamic related information on the basis of the second vibration information determining an aortic valve opening time of the supine subject on the basis of the first hemodynamic related information, and determining a pulse wave arrival time of the supine subject on the basis of the second hemodynamic related information; and determining an aortic Pulse Wave Transit Time of the supine subject on basis of the aortic valve opening time and the pulse wave arrival time.

Preferably, the first fiber-optic sensor or the second fiber-optic sensor comprise: an optical fiber, disposed substantially in a plane; a light source, coupled with one end of one or more optical fibers; a receiver, coupled to the other end of one optical fiber, and configured to sense changes in the intensity of light transmitted through the optical fiber; and a mesh layer, composed of meshes with openings; the mesh layer is in contact with the surface of the optical fiber.

Preferably, the step of generating first hemodynamic related information on the basis of the first vibration information, and generating second hemodynamic related information on the basis of the second vibration information by one of more processors, further comprises step of: filtering and scaling the first vibration information and the second vibration information to generate the first hemodynamic related information and the second hemodynamic related information.

Preferably, the step of determining an aortic valve opening time of the supine subject on the basis of the first hemodynamic related information, further comprises steps of: performing a second-order differential calculation on the first hemodynamic related information; performing a feature search to a waveform of the first hemodynamic related information after the second-order differential calculation to determine the highest peak in a cardiac cycle; and determining the aortic valve opening time of the supine subject based on the highest peak.

Preferably, the one or more processors are configured to execute the steps of: further comprising steps of: acquiring a distance between the first fiber-optic sensor and the second fiber-optic sensor in a body height direction to generate an aortic pulse wave conduction distance; and determining an aortic Pulse Wave Velocity on the basis of the aortic pulse wave conduction distance and the aortic Pulse Wave Transit Time.

Preferably, the one or more processors are configured to execute step of: sending at least one of the aortic Pulse Wave Transit Time and the aortic Pulse Wave Velocity to one or more output device, by the one or more processors.

At another aspect, a device provided in the present invention, comprises: a main body, used for a subject to lie down, comprising an upper cover and a lower cover, and having a back area and a waist area; a first fiber-optic sensor, being placed in the back area of the main body and used for acquiring first vibration information of the supine subject; and a second fiber-optic sensor group, comprising two or more fiber-optic sensors, being placed in the waist area of main body and used for acquiring second vibration information of the supine subject; wherein the upper cover and lower cover together enclose the first fiber-optic sensor and the second fiber-optic sensor group therein.

Preferably, the device comprises a neck pillow; the neck pillow is set on the upper cover, and used for supporting the neck of the supine subject whereby the subject can be located on the measuring position.

Preferably, the device comprises shoulder stops; the shoulder stops are set on the upper cover for the shoulder of the supine subject to abut against whereby the subject can be located on the measuring position.

Preferably, the main body comprises a lower limb area; the device comprises foot stops; the foot stops are set on the lower limb area of the upper cover for the feet or calves of the supine subject to abut against whereby the subject can be located on the measuring position.

Preferably, the upper cover of the main body is configured as a three-dimensional structure, and defines a body-contour recess whereby the supine subject can be located on the measuring position.

Preferably, two or more fiber-optic sensors of the second fiber-optic sensor group are configured to arrange along the longitudinal axis of the main body.

Preferably, the first fiber-optic sensor or the second fiber-optic sensor comprise: an optical fiber, disposed substantially in a plane; a light source, coupled with one end of one or more optical fibers; a receiver, coupled to the other end of one optical fiber, and configured to sense changes in the intensity of light transmitted through the optical fiber; and a mesh layer, composed of meshes with openings; the mesh layer is in contact with the surface of the optical fiber.

### Advantages

### BRIEF DESCRIPTION OF THE DRAWINGS

In order to explain more apparent to the technical solution of the embodiments of the present invention, a brief description to the drawings conjunct in the description of the embodiment is given below. Obviously, the drawings described below are used in only some embodiments of the invention. For those of ordinary skill in the art, without creative work, the present invention can also be applied to other similar embodiments based on these drawings. Unless it is obvious from the language environment or otherwise stated, the same reference numerals in the figures represent the same structure or steps.

FIG. 1 is a schematic diagram of a pulse wave conduction parameter measurement system in accordance with some embodiments of the present invention;

FIG. 2 is a schematic diagram of the principle of pulse wave generation;

FIG. 3 is a schematic diagram of the measurement principle of aortic pulse wave conduction parameters;

FIG. 4 is a block diagram of a computing device in accordance with some embodiments of the present invention;

FIG. 5 is a schematic diagram of a sensor device in accordance with some embodiments of the present invention;

FIG. 6 is a schematic diagram illustrating a position of the sensor device in accordance with some embodiments of the present invention;

FIG. 7 is a flowchart of a pulse wave conduction parameter measurement method in accordance with some embodiments of the present invention;

FIG. 8 is a signal waveform of a subject in accordance with some embodiments of the present invention;

FIG. 9 is a schematic diagram of the sensor device in accordance with some embodiments of the present invention;

FIG. 10 is a schematic diagram of a positioning indicator in accordance with some embodiments of the present invention; and

FIG. 11 is a schematic diagram of a positioning indicator in accordance with other embodiments of the present invention.

### DETAILED DESCRIPTION OF THE INVENTION

As used in the description and claims, the singular form “a”, “an” and “the” include both singular and plural references unless the context clearly dictates otherwise. Generally, the term “comprising” or “comprises” is intended to mean the steps or elements that have been clearly identified, and these steps or elements do not constitute an exclusive list, and the method or device can also include other steps or elements.

FIG. 1 is a schematic diagram of a pulse wave conduction parameter measurement system 100 in some embodiments of the present invention. As shown in FIG. 1, the pulse wave conduction parameter measurement system 100 can comprise a sensor device 101, a network 103, a server 105, a storage device 107, and an output device 109.

The sensor device 101 can be configured to acquire vibration information of the subject 102. In some embodiments, the sensor device 101 can be a vibration sensor, such as one or more of: an acceleration sensor, a speed sensor, a displacement sensor, a pressure sensor, a strain sensor, a stress sensor, or sensors that convert physical quantities equivalently based on acceleration, speed, displacement, or pressure (such as electrostatic sensors, inflatable micro-motion sensors, radar sensors, etc.). In some embodiments, the strain sensor can be an optical strain sensor. In some embodiments, the sensor device 101 can further include a temperature sensor, such as an infrared sensor, to obtain a body temperature of the subject. The sensor device 101 can be configured to be placed in various types of beds such as a medical bed or a nursing bed where the subject 102 is located. The subject 102 can be a vital body for vital signs monitoring. In some embodiments, the subject 102 can be a hospital patient or a caregiver, such as an elderly person, a prisoner, or other people. The sensor device 101 can transmit the acquired vibration information of the subject 102 to the server 105 through the network 103 for subsequent processing. In some embodiments, the vibration information obtained by the sensor device 101 can be processed to calculate the vital signs of the subject, such as heart rate, respiration rate, body temperature, and the like. In some embodiments, by processing the vibration information obtained by the sensor device 101, the pulse wave conduction parameters of the subject, such as the Pulse Wave Transit Time (PTT) and Pulse Wave Velocity PWV, can be calculated. The sensor device 101 can also transmit the acquired vibration information to the output device 109 for output, for example, for showing waveforms of the vibration information on a display. The sensor device 101 can also transmit the acquired vibration information of the subject 102 to the storage device 107 through the network 103 for storage. For example, the system 100 can comprises multiple sensor devices, and the vibration information of multiple subjects acquired by the multiple sensor devices can be transmitted to the storage device 107 to be stored as part of customer data.

The network 103 can perform information exchange. In some embodiments, the components of the pulse wave conduction parameter measurement system 100 (that is, the sensor device 101, the network 103, the server 105, the storage device 107, and the output device 109) can send or receive information between each other through the network 103. For example, the sensor device 101 can send the acquired vital signs of the subject 102 to the storage device 107 via the network 103 for storage. In some embodiments, the network 103 can be a single network, such as a wired network or a wireless network, or a combination of multiple networks. The network 103 can include, but is not limited to, LAN, WAN, a shared network, a dedicated network, and the like. The network 103 can include a variety of network access points, such as wireless or wired access points, base stations or network access points, through which other components of the pulse wave conduction parameter measurement system 100 can connect to the network 103 and send information via the network.

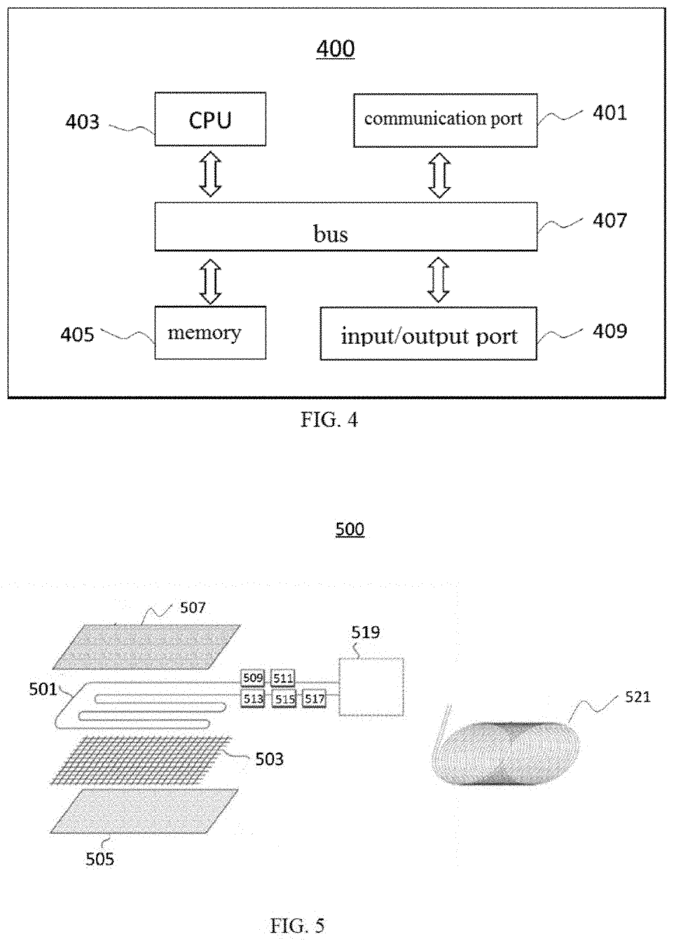

The server 105 is configured to process information. For example, the server 105 can receive the vibration information of the subject 102 from the sensor device 101, extract hemodynamic related signals from the vibration information, and further process the hemodynamic related signals to obtain the pulse wave conduction parameters of the subject 102. In some embodiments, the server 105 can be a single server or a server group. The server group can be clustered or distributed (that is, the server 105 can be a distributed system). In some embodiments, the server 105 can be local or remote. For example, the server 105 can access data stored in the storage device 107, the sensor device 101, and/or the output device 109 through the network 103. For another example, the server 105 can be directly connected to the sensor device 101, the storage device 107, and/or the output device 109 for data storage. In some embodiments, the server 105 can also be a cloud server, which can include, but is not limited to, public cloud, private cloud, hybrid cloud, and the like. In some embodiments, the server 105 can be implemented on the computing device 400 shown in FIG. 4.

The storage device 107 is configured to store data and instructions. In some embodiments, the storage device 107 can include, but is not limited to, Random Access Memory, Read Only Memory, Programmable Read-Only Memory, and the like. The storage device 107 can be a device for storing information by means of electrical energy, magnetic energy, and optical means, such as hard disks, floppy disks, magnetic core memories, CDs, DVDs, and the like. The storage devices mentioned above are just some examples, and the storage device 107 is not limited to these. The storage device 107 can store the vibration information of the subject 102 acquired by the sensor device 101, and can also store data from the vibration information processed by the server 105, such as vital signs (respiration rate, heart rate) of the subject 102. In some embodiments, the storage device 107 can be a component of the server 105.

The output device 109 is configured to output data. In some embodiments, the output device 109 can output the vital signs after being processed by the server 105, and the output manners include, but not limited to, one or more of: graphic display, digital display, voice broadcast, and braille display. The output device 109 can be one or more of: a display, a mobile phone, a tablet computer, a projector, a wearable device (watch, earphone, glasses, etc.), a braille display, and the like. In some embodiments, the output device 109 can display vital signs (such as respiration rate, heart rate, etc.) of the subject 102 in real time. In other embodiments, the output device 109 can display a report in non-real time, which is the measurement results of the subject 102 within the preset time period, such as the user's heart rate and the respiratory rate monitoring per minute during the sleeping period. In some embodiments, the output device 109 can also output early warning prompts, including but not limited to a sound alarm, a vibration alarm, and a screen display alarm, etc. For example, the subject 102 can be a patient being monitored, the output device 109 can be a display screen in a nurse's station, and the results displayed by the output device 109 can be real-time heart rate, real-time respiration rate, etc. When the heart rate or the respiration rate is abnormal (for example, exceeding a threshold or occurring a significant change during a preset time period), the output device 109 can emit an alarm sound to remind the medical staff, and the medical staff can rescue the patient in time. In other embodiments, the output device 109 can be a communication device (such as a mobile phone) carried by the doctor. When the vital signs of the subject 102 are abnormal, one or more output devices 109 carried by one or more doctors can receive the warning information, the warning information can be pushed according to the distance between the terminal device and the subjective 102.

It should be understood that the application scenarios of the system and method of the present invention are merely some examples or embodiments of the present invention. For those of ordinary skill in the art, they can also apply to other similar scenarios based on the drawings. The pulse wave conduction parameter measurement system 100 can be used in a family scene, and the sensor device 100 can be placed on an ordinary family bed, when the subject 102 (such as an elderly person, a person suffering from cardiovascular disease, or a person in a postoperative recovery period) sleeping at night, the sensor device 101 can acquire the subject's vibration information continuously or in a predetermined or required manner, and then send the vibration information through the network 103 (the vibration information can be sent in real time, or at a predetermined time, such as all the data of the previous night is sent at the next morning) to the cloud server 105 for processing. The cloud server 105 can send the processed information (such as heart rate per minute, respiratory rate per minute, aortic PWV) to the terminal 109. The terminal 109 can be a computer of the subject's family doctor, the family doctor can evaluate the physical condition and recovery of the subject 102 based on the processed information.

It should be noted that the above described embodiments should not be regarded as the only embodiments of the present invention. Obviously, for those skilled in the art, after understanding the content and principle of the present invention, it is possible to make various amendments or changes in form or in details without departing from the spirit and principle of the present invention, those amendments or changes are still within the protection scope of the claims of the present invention. In some embodiments, the server 105, the storage device 107 and the output device 109 can be implemented in a single device to achieve their respective functions. For example, the pulse wave conduction parameter measurement system 100 can include a sensor device and a computer. Where, the sensor device can be directly connected to the computer through a cable or through a network. The computer can implement all the functions of the server 105, the storage device 107 and the output device 109, and perform data processing, storage, display and other functions. In other embodiments, the pulse wave conduction parameter measurement system 100 can include a sensor device and an integrated circuit. The integrated circuit is integrated in the sensor device (for example, integrated in a mat). The integrated circuit is connected to a display screen to achieve the functions of the server 105 and the storage device 107 mentioned above with the display screen used as the output device 109, so as to realize the functions of data processing, storage and display.

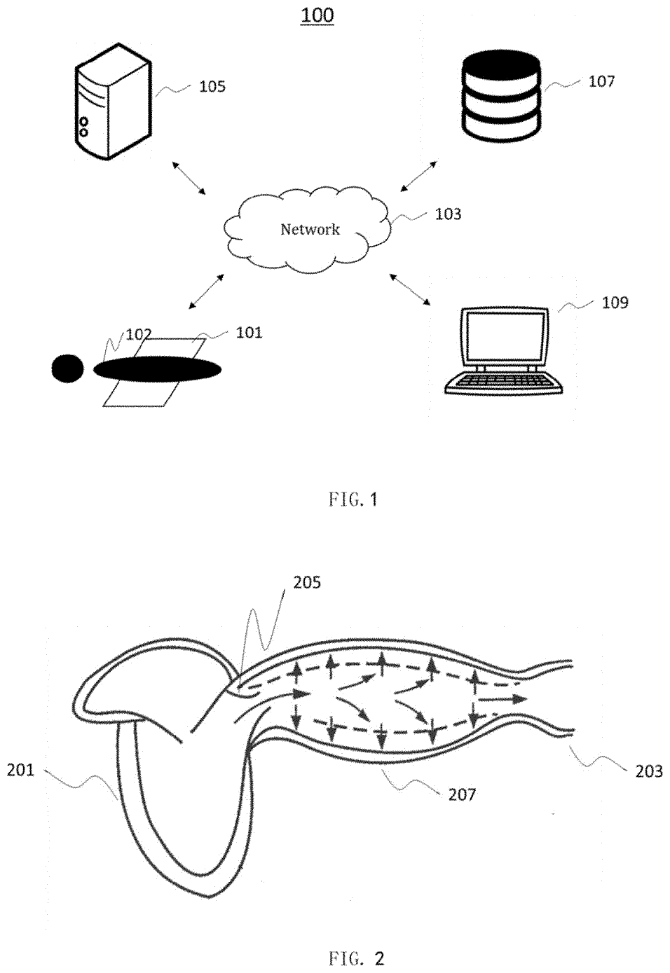

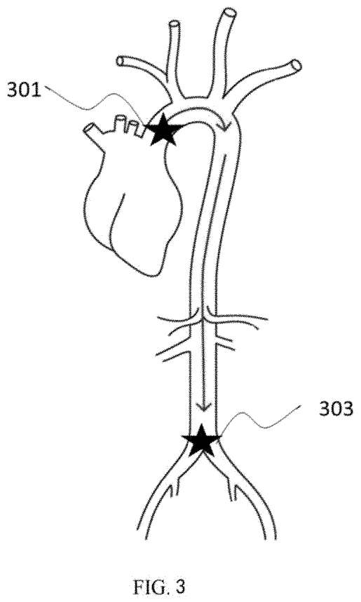

FIG. 2 is a schematic diagram illustrating the principle of pulse wave. As shown in FIG. 2, the left ventricle 201 and the aorta 203 are connected by the aortic valve 205. With the contraction of the left ventricle 201 to a certain pressure value, the aortic valve 205 opens (Aortic Valve Opening, AVO), and blood is injected from the left ventricle 201 into the aorta 203. Since the blood vessel is elastic, the blood will expand the aorta wall when injected into the aorta, and this pulse will propagate along the aortic wall, forming a pulse wave 207. Hemodynamics studies dynamics of blood flow in the cardiovascular system. It is based on blood flow and blood vessel wall deformation. The generation and propagation of pulse waves are related to blood flow and blood vessel wall deformation, which relates to hemodynamic research. The propagation velocity of the pulse wave 207 along the aorta is related to the elasticity of the aorta 203, therefore, the Pulse Wave Velocity PWV can be used to assess the degree of vascular stiffness.

FIG. 3 is a schematic diagram of the measurement principle of aortic pulse wave conduction parameters. As shown in FIG. 3, the aorta can be divided into ascending aorta, aortic arch, and descending aorta. The ascending aorta starts from the aorta of the left ventricle and continues to the aortic arch obliquely to the upper right side. The brachiocephalic artery, the left common carotid artery, and the left subclavian artery arise from the aortic arch; and the brachiocephalic artery are divided into the right common carotid artery and the right subclavian artery behind the right sternoclavicular joint. The aortic arch is connected to the ascending aorta, arched at the back of the sternum stem to the left and rear, and the arch is moved to the left and back to the lower border of the fourth thoracic vertebra as the descending aorta. The descending aorta is the longest segment of the aorta. It splits into the left and right common iliac arteries at the fourth lumbar vertebra. It can be seen that the pulse wave of the aortic segment starts from the origin of the aortic 301 and is conducted along the aorta to the bifurcation 303 of the aorta and the left and right common iliac arteries. Therefore, the distance along the path of the aorta from the origin of the aorta 301 to the bifurcation 303 of the aorta and the left and right common iliac arteries is taken as the aortic pulse wave conduction distance, the time for the pulse wave propagating from Point 301 to Point 303 is taken as the aortic Pulse Wave Transit Time, and the ratio of the aortic pulse wave conduction distance to the transit Time is taken as the aorta Pulse Wave Velocity (aortic PWV, aPWV).

FIG. 4 is a block diagram of a computing device 400 in some embodiments of the present invention. In some embodiments, the server 105, the storage device 107, and/or the output device 109 of FIG. 1 can be implemented by the computing device 400. For example, the server 105 can be implemented by the computing device 400 and configured to perform the functions of the server 105 described in this invention. In some embodiments, the computing device 400 can be a dedicated computer. For ease of description, only one server is shown in FIG. 1. For those of ordinary skill in the art, it should be understood that calculation functions related to pulse wave conduction parameter measurement can also be implemented by multiple computing devices with similar functions so as to distribute the calculation load.

The computing device 400 can include a communication port 401, a processor (Central Processing Unit, CPU) 403, a memory 405, and a bus 407. The communication port 401 is configured to exchange data with other devices through the network. The processor 403 is configured to perform data processing. The memory 405 is configured to store data and instructions, and the memory 405 can be a Read-Only Memory ROM, a Random Access Memory RAM, a hard disk, and other forms of memory. The bus 407 is configured to perform data communication in the computing devices 400. In some embodiments, the computing device 400 can further include an input/output port 409which is used for data input and output. For example, other persons can use an input device (such as a keyboard) to input data to the computing device 400 through the input/output port 409. The computing device 400 can also output data to an output device such as a display through the input/output port 409.

It should be understood, the easy of description, only one processor 403 is described here. It should be understood that the computing device 400 can include multiple processors, and the operations or methods executed by one processor 403 can be jointly or separately executed by multiple processors. For example, one processor 403 described in the present invention can perform step A and step B. It should be understood that step A and step B can be executed jointly or separately by multiple processors. For example, the first processor executes step A, and the second processor executes step B, or the first processor and the second processor jointly execute steps A and B.

FIG. 5 is a schematic diagram of a fiber-optic sensor device 500 in some embodiments of the present invention. As shown in FIG. 5, the fiber-optic sensor device 500 is a strain sensor. When an outside force is applied to the fiber-optic sensor device 500, for example, placing the fiber-optic sensor device 500under the lying human body, when the subject is at rest, the human body's respiration and heartbeat will cause the human body to vibrate. The vibration of the human body can cause the bending of the optical fiber 501. The bending of the optical fiber changes the parameters of the light traveling through the optical fiber, such as light intensity. The changes in an intensity of light after processing can be used to represent the body's vibration.

The fiber-optic sensor device 500 can include an optical fiber 501, a mesh layer 503, an upper cover 507, and a lower cover 505. Where one end of the optical fiber 501 is coupled to a light source 509, which can be an LED light source. The light source 509 is coupled to a light source driver 511, which is configured to control the switch and energy level of the light source. The other end of the optical fiber 501 is coupled to a receiver 513.The receiver 513 is configured to receive the optical signal transmitted through the optical fiber 501.The receiver 513 is coupled to an amplifier 515, and the amplifier 515 is coupled to an Analog-to-Digital Converter 517, which can convert the received optical signal into a digital signal. The light source driver 511 and the Analog-to-Digital Converter 517 are coupled to a control and processing module 519. The control and processing module 519 is configured for signal control and signal processing. For example, the control and processing module 519 can control the light source driver 511 to drive the light source 509 emitting light; and the control and processing module 519 can also receive data from the Analog-to-Digital Converter 517, and process the data adapted for wireless or wired network data transmission so that the processed data can be transmitted via the wireless or wired network to other devices, such as the server 105, the storage device 107, and/or the output device 109 in FIG. 1. The control and processing module 519 can also control the sampling rate of the Analog-to-Digital Converter 517 so that it has different sampling rates according to different requirements. In some embodiments, the light source driver 511, the receiver 513, the amplifier 515, the Analog-to-Digital Converter 517, and the control and processing module 519 can be combined into one module to perform all functions.

The optical fiber 501 can be a multi-mode optical fiber, or can be a single-mode optical fiber. The optical fibers can be arranged in various shapes, such as a serpentine structure, referring to the shape of 501 as shown in FIG. 5. In some embodiments, the optical fibers 501 can be arranged in a U-shape. In some embodiments, the optical fibers 501 can be arranged in a looped structure. Referring to 521, the looped structure is formed by one optical fiber arranged into a plurality of equal-sized loops disposed substantially in a plane, where each loop within the looped structure is partially overlapping yet laterally offset from neighboring loops. Each of the loops can form a substantially parallelogram structure (such as a rectangle, a square, etc.) with rounded edges without sharp bends. In some embodiments, each of the loops forms a circle or other ellipse. In some other embodiments, each of the loops forms a matching irregular shape without sharp bending.

The mesh layer 503 is made of any suitable material with through holes arranged in a repeating pattern. In some embodiments, the mesh is formed of woven fibers, such as polymer fibers, natural fabric fibers, composite fabric fibers, or other fibers. When the fiber-optic sensor device 500 is placed under the subject's body, the subject will apply an outside force to the fiber-optic sensor device 500. The mesh layer 503 can disperse the outside force that would have been applied to a certain point of the fiber and distribute to around the point of the fiber. Micro-bending in the optical fiber 501 causes changes in the parameter (such as the intensity) of light transmitted in the optical fiber 501. The receiver 513 receives the residual light and changes in the amount of light are processed and determined by the control and processing module 519. The amount of bending of the optical fiber 510 under the application of outside force depends on the applied force, the diameter of the optical fiber, the diameter of the mesh fiber, and the size of the openings in the mesh. By balancing these parameters of the diameter of the optical fiber, the diameter of the mesh fiber, and the size of the openings in the mesh, when the external office is applied, the optical fiber will bend in different amount, which makes the fiber-optic sensor device 500 having different sensitivity to the outside force.

The upper cover 507 and the lower cover 505 can be made of silicone material, and are configured to surround the optical fiber 501 and the mesh layer 503, which can protect the optical fiber 501 and distribute the outside force so that the outside force is dispersed around the force application point. The upper cover 507, the optical fiber 501, the mesh layer 503, and the lower cover 505 can be bonded as a whole, for example, glued together with a silicone adhesive, so that the fiber-optic sensor device 500 forms a sensor pad. The width and/or length of the sensor pad can be different according to different arrangements of the optical fibers. When the looped structure is used, the width of the sensor pad can be 6 cm or more than 6 cm other suitable widths such as 8 cm, 10 cm, 13 cm or 15 cm. The length of the sensor pad can be different according to different usage scenarios. For example, for people in a normal height range, the length of the sensor pad can be between 30 cm and 80 cm, such as 50 cm, or other suitable sizes; the length of 45 cm is suitable for most people. In some embodiments, the thickness of the sensor pad can be 1-50 mm, preferably, 3 mm. In some embodiments, the width and length of the sensor pad can be other sizes, and sensors of different sizes can be selected according to different test subjects. For example, test subjects can be divided into groups according to age, height, and weight. Different groups use the sensors of different sizes. In some embodiments, when the optical fiber is arranged in a U-shape, the width of the sensor pad can also be less than 6 cm, for example, 1 cm, 2 cm, or 4 cm.

In some embodiments, the fiber-optic sensor device 500 can further comprise an outer cover (not shown in FIG. 5) enclosing the upper cover 507, the mesh layer 503, the optical fiber 501 and the lower cover 505. The outer cover can be made of oil-resistant and water-repellent materials, such as hard plastic. In other embodiments, the fiber-optic sensor device 500 can further comprise a support structure (not shown in FIG. 5). The support structure can be a rigid structure, such as cardboard, hard plastic board, wood board, etc. The support structure can be placed between the optical fiber 501 and the lower cover 505 so as to provide a support for the optical fiber 501. When an outside force is applied to the optical fiber 501, the support structure can make the deformation of the optical fiber rebound faster and the rebound time shorter, so that the optical fiber can capture higher frequency signal.

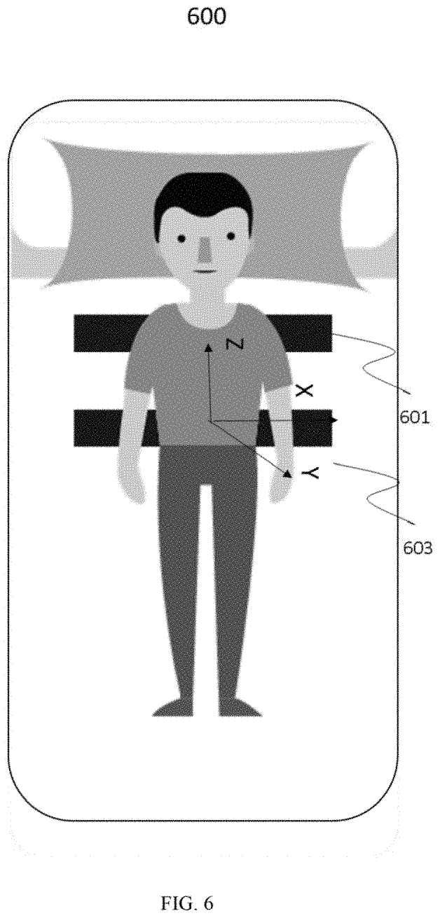

FIG. 6 shows positions of the sensor device in some embodiments of the present invention. As shown in FIG. 6, the sensor device 600 can include, but not limited to, a fiber-optic sensor 601 and a fiber-optic sensor 603. In some embodiments, the fiber-optic sensor 601 and the fiber-optic sensor 603can adopt the fiber-optic sensor device 500.

In order to clearly illustrate the positions and relationships of the body parts and the relationship between the positions of the sensor device and the body parts in the present invention, the anatomical coordinate system is introduced here. The standard anatomical position of the human body comprises an upright position and a supine position. Take the supine position as an example, as shown in FIG. 6, the X-axis is the median horizontal axis, the Y-axis is the median sagittal axis, and the Z-axis is the median vertical axis. The origin O is located at the midpoint of the upper edge of the phalanx syndesmosis. The YZ plane is the median sagittal plane, which divides the human body into left and right parts, the XZ plane is the median coronal plane, which divides the human body into front and back parts, and the XY plane is the origin transverse plane, which divides the human body into upper and lower parts. The front, back, upper, lower, left, and right parts of the human body described in the present invention are described on the basis of the anatomical coordinate system.

In some embodiments, the fiber-optic sensor 601 can be placed under the back of the subject 102 corresponding to the origin of the aorta, approximately under the back corresponding to the fourth thoracic vertebra of the human body. The fiber-optic sensor 603 can be placed under the back of the subject 102 corresponding to the bifurcation of the aorta and the left and right common iliac arteries, approximately under the back corresponding to the fourth lumbar vertebra of the human body. According to different subjects and/or different application scenarios, the length and width of the fiber-optic sensors 601 and 603 can be selected according to actual needs. For example, the length (along the X-axis) can be between 30 cm and 80 cm, and the width (along The Y-axis) can be between 1 cm and 20 cm, or it can be other suitable sizes. In some embodiments, the fiber-optic sensors 601 and 603 are two independent sensors, and their positions can be changed. For example, different heights of subjects cause different lengths of aortic segments, so the distance between the fiber-optic sensor 601 and the fiber-optic sensor 603 can be adjusted according to the height of the subject. In some embodiments, the sensor device 600 can comprise a body for the subject to lie down. For example, the body can be a cushion, the cushion includes an upper cover and a lower cover, and the upper cover and the lower cover are bonded into a whole. The cushion can wrap the fiber-optic sensors 601 and 603 inside the space formed by the upper cover and the lower cover, and fix their positions. The distance between the fiber-optic sensor 601 and the fiber-optic sensor 603 can be preset according to actual needs. In an exemplary embodiment, the distance can be between 20cm and 80cm, or other suitable distance. The shape and size of the sensor device 600 can be selected according to actual needs. For example, the sensor device 600 can have quadrilateral, circular or other suitable shapes. The sensor device 600 can be configured in different sizes according to people in a normal height range. For example, the size suitable of the sensor device for people in a height of 155 cm-160 cm is 40 cm, which is set as S size, and the size for people group in a height of 161 cm-170 cm can increase a certain distance based on the S size, for example increase 3 cm. In other embodiments, the fiber-optic sensors 601 and 603 are enclosed inside the cushion, and the position of one of the fiber sensors can be fixed (for example, the fiber sensor 601 is fixed), and a space is defined inside the cushion for moving the other sensor (such as fiber-optic sensor 603) to change the position thereof. For example, a sliding track is arranged inside the cushion, and the fiber-optic sensor 603 is set on the track. A control device is provided outside the cushion so that the operator can control the movement of the fiber-optic sensor 603 using the control device. For example, the control device is a handle. The movement of the fiber-optic sensor 603 can be manually controlled. As another example, the control device is a switch; when the control device is in a turn-on state, the fiber-optic sensor 603 automatically moves toward or away from the fiber-optic sensor 601 at a preset speed; when the control device is in the turn-off state, the fiber-optic sensor 603 becomes stationary. Where the outside of the cushion can be provided with scale marks, for example, along the sliding track, so that the operator can directly read the distance between the fiber-optic sensor 601 and the fiber-optic sensor 603.

It should be understood that the application scenarios of the device, system, and method of the present invention are only some examples or embodiments. For those of ordinary skill in the art, without creative work, the present invention can be applied to other similar scenarios based on the drawings. For example, the sensor device 101 cannot be limited to the form of the fiber-optic sensor device 500 and the sensor device 600, and thus is applicable to other scenarios.

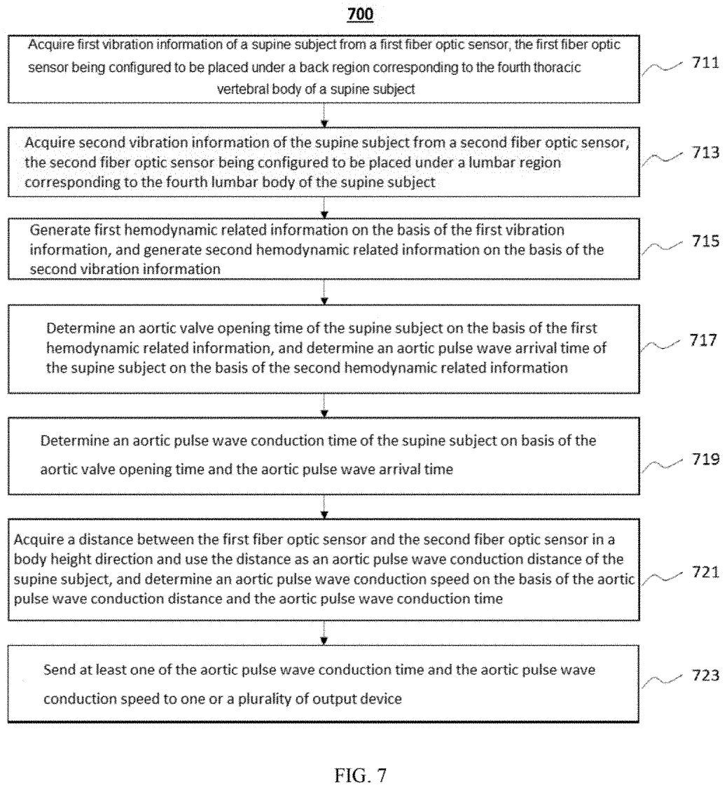

FIG. 7 illustrates a flowchart of a pulse wave conduction parameter measurement method in some embodiments of the present invention. In some embodiments, the method 700 can be implemented by the pulse wave conduction parameter measurement system 100 shown in FIG. 1. For example, the method 700 can be stored as an instruction set in the storage device 107and executed by the server 105. The server 105 can be implemented by means of the computing device 400.

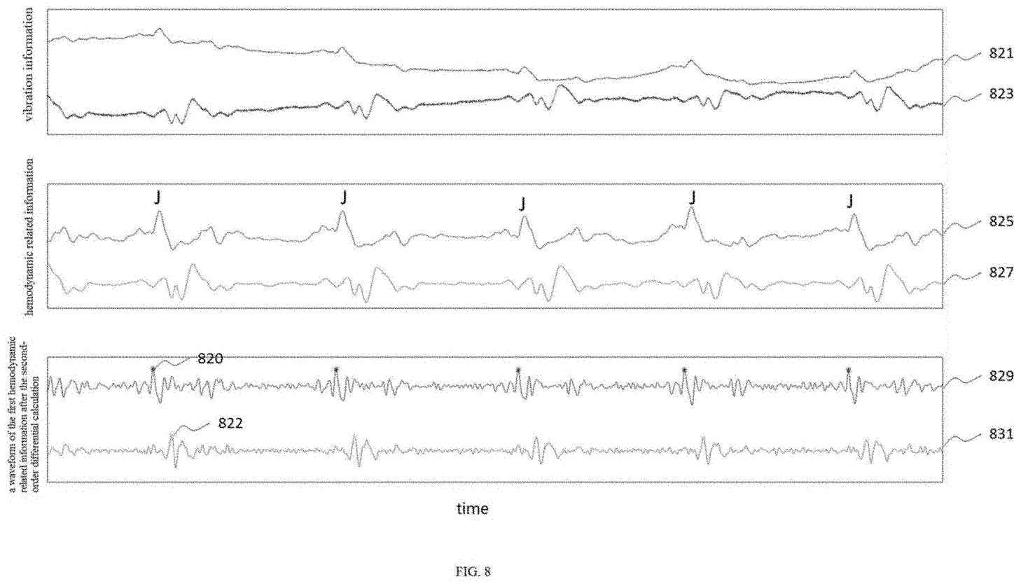

Step 711, acquiring first vibration information of a supine subject from a first fiber-optic sensor by the processor 403, the first fiber-optic sensor being placed under a back section corresponding to the fourth thoracic vertebra of a supine subject. In some embodiments, the supine subject can be a hospital patient or a caregiver, etc., in a supine position, lying on the sensor device 600. The first fiber-optic sensor can be the fiber-optic sensor 601 of the sensor device 600, and the fiber-optic sensor 601 is placed under the back section corresponding to the origin of the aorta of the supine subject, approximately under the back section corresponding to the fourth thoracic vertebra. The first vibration information of the supine object can comprise one or more of: body vibration information caused by breathing, body vibration information caused by contraction and relaxation of the heart, body vibration information caused by blood vessel wall deformation, and body movement information of the human body. Body vibration information caused by contraction and relaxation of the heart can include body vibration information caused by the contraction and relaxation of the heart itself, as well as body vibration information caused by blood flow caused by contraction and relaxation of the heart, such as body vibration information caused by blood flowing in the aortic arch due to heart's ejection. Body vibration information caused by blood vessel wall deformation, can be caused by pulse wave propagation along blood vessels, where heart's ejection causes the aortic wall to expand to form a pulse wave. The body movement information can be caused by the body movement such as leg bending, leg raising, turning over, shaking, etc. Specifically, breathing will cause the whole body, especially the body sections corresponding to the thorax and abdomen, to vibrate rhythmically. The contraction and relaxation of the heart will also cause the whole body, especially the body around the heart, to vibrate. The left ventricle pumps blood to the aorta, the blood will push against the aortic arch at the moment; and the heart itself and the connected large blood vessels as a whole will also undergo a series of movements. The farther the body part is from the heart, the weaker the vibration will be. The pulse wave propagating along the blood vessels will cause body vibration according to the blood vessels; the thinner the blood vessels and the farther away from the heart, the weaker the body vibration. Therefore, when the sensor is placed under different positions of the body, the vibration information acquired by the sensor is the aforementioned body vibration information detected at this position, and when the position is different, the body vibration information acquired is also different. The aorta is the largest artery in the human body, originating from the left ventricle of the heart and extending down to the thoracic cavity and abdomen. Therefore, when the fiber-optic sensor 601 is placed under the back section corresponding to the fourth thoracic vertebra of the subject, where is near the heart, therefore, all or part of the body vibration information can be acquired and generate the first vibration information. As shown in FIG. 8, a curve 821 is a waveform of the first vibration information of a subject acquired by the fiber-optic sensor 601 placed under the back section corresponding to the fourth thoracic vertebra of the subject in an embodiment of the present invention. Where, the horizontal axis represents time, and the vertical axis represents the first vibration information of the subject after normalization processing, which is dimensionless.

Step 713, acquiring second vibration information of the supine subject from a second fiber-optic sensor by the processor 403, the second fiber-optic sensor being placed under a lumbar section corresponding to the fourth lumbar vertebra of the supine subject. In some embodiments, the second fiber-optic sensor can be the fiber-optic sensor 603 in the sensor device 600. The fiber-optic sensor 603 is placed under the waist position corresponding to the bifurcation of the descending aorta and the left and right common iliac arteries of the supine subject, approximately under the waist section corresponding to the fourth lumbar vertebra. Since the fiber-optic sensor 603 is placed according to the end of the subject's aorta, within the body section of the abdominal cavity, the acquired second vibration information can comprise body vibration information caused by breathing, body vibration information caused by contraction and relaxation of the heart, and body vibration information caused by pulse wave propagation along blood vessels. As shown in FIG. 8, the curve 823 is a waveform of the second vibration information of a subject acquired by a fiber-optic sensor 603 placed under the waist section corresponding to the fourth lumbar vertebra of the subject in an embodiment of the present invention, where, the horizontal axis represents time, and the vertical axis represents the second vibration information of the subject after normalization processing, which is dimensionless.

Step 715, generating first hemodynamic related information on the basis of the first vibration information, and generating second hemodynamic related information on the basis of the second vibration information, which can be performed by the processor 403. Hemodynamics studies dynamics of blood flow in the cardiovascular system. It is based on blood flow and blood vessel wall deformation. The “hemodynamic related information” described in this invention refers to any information related to hemodynamics, which can include, but not limited to, one or more of: information related to blood flow generation (for example, heart's ejection caused by the contraction and relaxation of the heart), and blood flow-related information (such as cardiac output CO, left ventricular ejection impacting the aortic arch), blood pressure-related information (such as systolic arterial pressure, diastolic blood pressure, mean arterial pressure), or blood vessel-related information (For example, vascular elasticity). Pulse wave conduction parameters, such as Pulse Wave Velocity, are not only related to blood vessel elasticity, but also to the contraction and relaxation of the heart, and left ventricular ejection impacting the aortic arch. Therefore, the measurement of pulse wave conduction parameters involves the acquisition of hemodynamic related information. In some related literatures, Ballistocardiogram (BCG) signal is used to represent periodic motions of the human body caused by heart beating. In body vibration information obtained by the vibration sensor described in the present invention, the body vibration information caused by the contraction and relaxation of the heart can also be expressed as a BCG signal. The hemodynamic related information described in the present invention includes BCG signals. In some embodiments, on the basis of the first vibration information acquired by the fiber-optic sensor 601 in step 711, the first hemodynamic related information to be generated by the processor 403 can comprise: vibration information caused by left ventricular ejection impacting the aortic arch, and vibration information caused by blood vessel wall deformation (that's, vibration information caused by pulse wave propagation along blood vessels). On the basis of the second vibration information acquired by the fiber-optic sensor 603 in step 713, the second hemodynamic related information to be generated by the processor 403 can comprise vibration information caused by pulse wave propagation along the blood vessel. As shown in FIG. 8, the curve 825 is a time-domain waveform of the first hemodynamic related information generated by the processor 403 on the basis of the first vibration information of the curve 821, and the curve 827 is a time-domain waveform of the second hemodynamic related information generated by the processor 403 on the basis of the second vibration information of the curve 823, and the horizontal axis represents time.

In some embodiments, generating first hemodynamic related information on the basis of the first vibration information, and generating second hemodynamic related information on the basis of the second vibration information by the processor 403. The first vibration information and/or the second vibration information include a variety of sub-vibration information (vibration information caused by breathing, vibration information caused by heart contraction, and vibration information caused by blood vessel wall deformation). The processor 403 can perform filtering in different frequency for different sub-vibration information. For example, the processor 403 can set the filtering frequency to below 1Hz for filtering the vibration information caused by breathing, and the processor 403 performs filtering including but not limited to one or more of: low-pass filtering, band-pass filtering, IIR (Infinite Impulse Response) filtering, FIR (Finite Impulse Response) filtering, wavelet filtering, zero-phase bidirectional filtering, and polynomial fitting and smoothing filtering, where the first and/or second vibration information can be filtered at least once. If the vibration information carries power frequency interference, a power frequency filter can used to filter power frequency noise. The processor 403 can filter the vibration information in the time domain or in the frequency domain. The processor 403 can also scale the filtered and denoised first/second vibration information according to the signal dynamic range to obtain the first/second hemodynamic related signal.

Step 717, determining an aortic valve opening time of the supine subject on the basis of the first hemodynamic related information, and determining a pulse wave arrival time of the supine subject on the basis of the second hemodynamic related information, which can be performed by the processor 403. The first hemodynamic related information can include the vibration information caused by the impact of the blood flow in the aortic arch when the left ventricle ejects blood, and the vibration information caused by the pulse wave propagating along the blood vessel. In a cardiac cycle, the aortic valve opens, the left ventricle ejects blood, and the time when blood enters the aorta is considered as the time point of pulse wave generation. At this moment, the blood flow ejected from the left ventricle will impact the aortic arch, causing the heart itself and its connected large blood vessels as a whole undergoes a series of movements, which causes movement to generate displacement in the body. Since the heart contracts and relaxes periodically, the displacement in the body also changes periodically, such kind of vibration information can be transmitted through the bones and muscles of the human body, and can be captured by the first fiber-optic sensor placed under the back section corresponding to the fourth thoracic vertebra of the supine subject. Since the time delay between the event of the aortic valve opening and the event of the sensor capturing the corresponding body vibration information is usually small, about within 10 ms, the time delay can be ignored in subsequent pulse wave conduction parameter measurement. That is, the time that the sensor captures the body vibration information caused by the aortic valve opening is used as the aortic valve opening time; or, a correction coefficient can be used to correct the actually-measured aortic valve opening time. The pulse wave propagates along the blood vessel, and the vibration is also conducted along the blood vessel, causing vibration of the body. Therefore, when the pulse wave reaches a certain position in the blood vessel, the vibration sensor at the position of the blood vessel can capture the vibration information. The second fiber-optic sensor under the waist section of the fourth lumbar vertebra of the supine subject can capture the vibration information of the pulse wave transmitted to the end of the aortic segment (i.e. the bifurcation of the descending aorta and the left and right common iliac arteries). Similarly, the time delay between the arrival time of the pulse wave and the second fiber-optic sensor capturing the corresponding body vibration information is relatively small. This time delay can be ignored in subsequent pulse wave conduction parameter measurement, that is, the time when the sensor captures the body vibration information caused by the pulse wave reaching the end of the arterial segment, is used as the pulse wave arrival time or, a correction coefficient can be used to correct the actually-measured pulse wave arrival time.

In some embodiments, determining the aortic valve opening time of the supine subject based. on the first hemodynamic related information, the processor 403 can perform the following steps. As shown in FIG. 8, the curve 825 is a time-domain waveform of the first hemodynamic related information, and the processor 403 can perform the step of: obtaining a curve 829 by a second-order differential calculation to the curve 825. The processor 403 can perform a step of: determining the aortic valve opening feature points by means of a feature search to the waveform 829. The features in the feature search can include, but not limited to, peaks, troughs, wave widths, amplitudes, the maximum value of the function, the minimum value of the function, maximums, minimums, etc. In some embodiments, the feature search to the curve 829 can use the peak search, with each cycle as a search range, the highest peak searched in a cycle is regarded as the aortic valve opening feature point, and the corresponding time is the aortic valve opening time. As shown by the curve 829 in FIG. 8, in the first complete cardiac cycle, point 820 is the feature point of aortic valve opening. In other embodiments, the processor 403 can also directly perform step of: determining the feature points of aortic valve opening via a feature search to the curve 825. For example, using a cardiac cycle as a search range, first search for the highest peak J, and then searching within the range before the time corresponding to the peak J, and searching for the minimum value of the function (AVO peak), which is regarded as the feature point of aortic valve opening, and the corresponding time is the aortic valve opening time.

In some embodiments, the processor 403 can perform the following steps of: determining the pulse wave arrival time of the supine subject on the basis of the second hemodynamic related information. As shown in FIG. 8, a curve 827 is a time-domain waveform of the second hemodynamic related information, and the processor 403 can perform the step of: obtaining a curve 831 by a second-order differential calculation to the curve 827.The processor 403 can perform a step of: determining the feature point of the pulse wave arrival by a feature search to the waveform 831. The features in the feature search can include, but not limited to, peaks, troughs, wave widths, amplitudes, the maximum value of the function, the minimum value of the function, maximums, minimums, etc. In some embodiments, the feature search of the curve 831 can use the peak search, with each cycle as a search range, the highest peak searched in a cycle is regarded as the feature point of the pulse wave arrival, and the corresponding time is the pulse wave arrival time. As shown by the curve 831 in FIG. 8, in the first complete cardiac cycle, the point 822 is the feature point of the pulse wave arrival.

In some embodiments, the processor 403 can perform other essentially equivalent digital signal processing methods, such as using polynomial fitting and smoothing filtering, to obtain information equivalent to performing second-order differential calculation.

In some embodiments, the first vibration information and the second vibration information of the supine subject are continuously acquired, and there can be data waveforms in one or several cardiac cycles that are different from the data waveforms of other cardiac cycles, where, the feature point of aortic valve opening and the feature point of pulse wave arrival in the cardiac cycle cannot be the highest peaks, and can be submerged. At this time, the data of the cardiac cycle can be discarded.

In some embodiments, the processor 403 can receive user input from one or more input devices to determine the aortic valve opening time and the pulse wave arrival time of the supine subject. For example, the external input parameters can be input by the medical staff to the processing device 400 through the input/output port 409 using an input device (for example, a mouse, a keyboard). Medical staff is trained to have the ability to judge feature points from the waveform of the vibration signal. For example, medical staff can manually analyze the waveform of curve 825, first select the highest peak in a cardiac cycle, and then search for the minimum value of the waveform in the same cycle range before the time corresponding to the highest peak, and mark as the aortic valve opening feature point and mark it using an input device, for example, select the feature points using a mouse. Therefore, the processor 403 can determine the input of the medical staff as the aortic valve opening feature point and automatically obtain its corresponding time as the aortic valve opening time.

Step 719, determining an aortic Pulse Wave Transit Time of the supine subject on basis of the aortic valve opening time and the pulse wave arrival time by the processor 403. In some embodiments, the processor 403 can obtain the difference between the aortic valve opening time and the pulse wave arrival time (by subtracting the aortic valve opening time from the pulse wave arrival time) in any one cardiac cycle as the aortic Pulse Wave Transit Time. In some embodiments, the processor 403 can select multiple cardiac cycles, for example 20 cardiac cycles, calculate the aortic Pulse Wave Transit Time (i.e., PTT1, PTT2 . . . PTT20) in each cardiac cycle, and then calculate the average value as aortic Pulse Wave Transit Time. In some embodiments, the processor 403 can select a fixed duration, such as 60 seconds, calculate the Pulse Wave Transit Time (i.e., PTT1, PTT2 . . . ) in each cardiac cycle within the duration, and calculate the average value as the Pulse Wave Transit Time. In other embodiments, the processor 403 can also automatically remove data whose Pulse Wave Transit Time is not within a reasonable range and use the average value of the remaining data as the Pulse Wave Transit Time. In other embodiments, the processor 403 can also calculate the Pulse Wave Transit Time in all cycles collected in the test, and calculate the average value thereof as the Pulse Wave Transit Time.

Step 721, acquiring a distance between the first fiber-optic sensor and the second fiber-optic sensor in a body height direction and using the distance as an aortic pulse wave conduction distance of the supine subject, and determining an aortic Pulse Wave Velocity on the basis of the aortic pulse wave conduction distance and the aortic Pulse Wave Transit Time, which can be performed by the processor 403. In some embodiments, the first fiber-optic sensor and the second fiber-optic sensor are independent devices, and the distance between the two sensors can be manually adjusted according to subjects of different heights. Here, the aortic pulse wave conduction distance can be measured manually. For example, medical staff can measure the distance between the first fiber-optic sensor and the second fiber-optic sensor along the height direction of the body using distance measuring tools such as a soft ruler, a ruler, or a line with scale, as the pulse wave conduction distance. In some embodiments, the distance between the first fiber-optic sensor and the second fiber-optic sensor can be fixed, and the distance between the two sensors as a fixed parameter will be transmitted to the processor 403 when the system is initialized. In some embodiments, the processor 403 can directly use the obtained distance between the first fiber-optic sensor and the second fiber-optic sensor in the height direction of the body as the aortic pulse wave conduction distance. In other embodiments, the obtained distance between the first fiber-optic sensor and the second fiber-optic sensor along the height direction of the body can be corrected by the processor 403, for example, by a correction coefficient, or by adding a constant, and then used as the aortic pulse wave conduction distance.

In other embodiments, the aortic pulse wave conduction distance can be estimated according to a formula. For example, the height, weight, age and other parameters of the test subject can be input via the input device of the system 100, and the pulse wave conduction distance of the test subject can be estimated according to the formula by the processor 403. For example, the processor 403 can estimate the length of the aorta of the test subject according to the following formula, which is the aortic pulse wave conduction distance:

L=a+b* (age)+c* (height)+d*(weight)

Where, L represents a length of the aorta in centimeters, age in years, height in centimeters, and weight in kilograms. Further, a represents a constant, and b, c, and d are coefficients. The values of a, b, c, d can be obtained by fitting calculation according to the actually-measured aortic length and the age, height, weight, etc. of each tester. In some embodiments, a can be −21.3, b can be 0.18, c can be 0.32, and d can be 0.08.

Step 723, sending at least one of the aortic Pulse Wave Transit Time and the aortic Pulse Wave Velocity to one or more output device, which can be performed by the processor 403.For example, the Pulse Wave Transit Time can be sent to the output device 109 in the system 100 for output. The output device 109 can be a display device, such as a mobile phone, which can display the Pulse Wave Transit Time in graphics or text. The output device 109 can be a printing device, which prints the measurement report of the pulse wave conduction parameters. The output device 109 can be a voice broadcast device, which outputs pulse wave conduction parameters in voice. In some embodiments, the processor 403 can send the Pulse Wave Transit Time and/or the Pulse Wave Velocity to an output device via a wireless network, for example, the output device is a mobile phone. In other embodiments, the processor 403 can directly send the Pulse Wave Transit Time and/or the Pulse Wave Velocity to the output device through a cable. For example, the output device is a display, which can be connected to the sensor device through a cable.

In some embodiments, the steps of the method 700 can be performed in order, in other embodiments, the steps of the method 700 can be performed not in order, or can be performed simultaneously. For example, steps of: step 719,determining an aortic Pulse Wave Transit Time on basis of the aortic valve opening time and the aortic pulse wave arrival time; step 721, Step 721, acquiring a distance between the first fiber-optic sensor and the second fiber-optic sensor in a body height direction and using the distance as an aortic pulse wave conduction distance of the supine subject, and determining an aortic Pulse Wave Velocity on the basis of the aortic pulse wave conduction distance and the aortic Pulse Wave Transit Time; and step 723, sending the aortic Pulse Wave Transit Time to one or more output device; can be performed simultaneously. In addition, without departing from the spirit and scope of the subject matter described herein, in some embodiments, one or more steps of the method 700 can be removed. For example, step 721 and/or step 723may not be performed. In other embodiments, other steps may be added to the method 700.

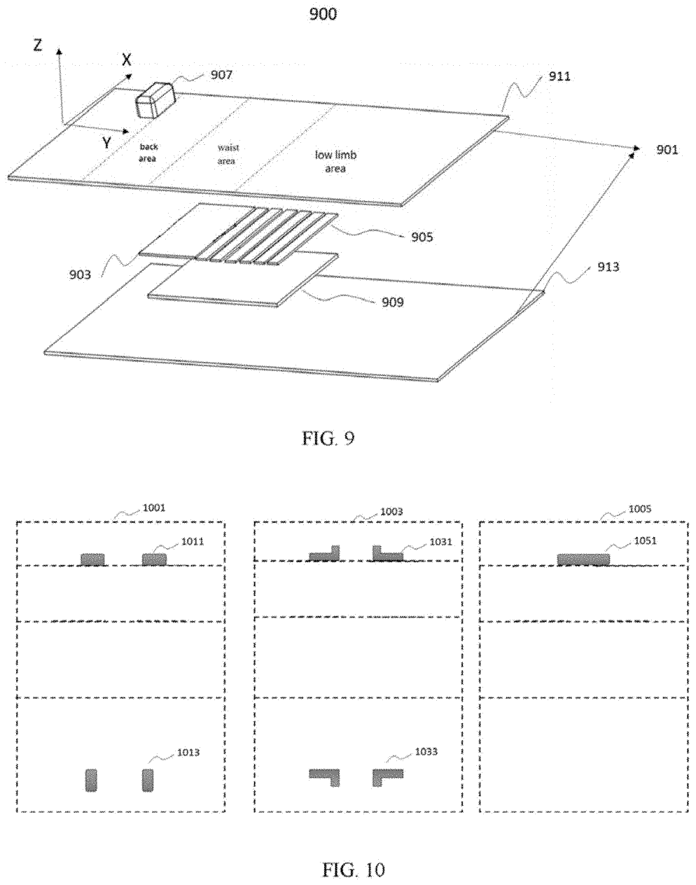

FIG. 9 is a schematic diagram of a sensor device in some embodiments of the present invention. As shown in FIG. 9, the sensor device 900 can include but not limited to a main body 901, a first fiber-optic sensor 903, a second fiber-optic sensor group 905, and a positioning indicator 907.

In order to clearly explain the positional relationship between the first fiber-optic sensor 903, the second fiber-optic sensor group 905, and the positioning indicator 907 and the positional relationship with the main body 901 in the present invention, the corresponding coordinates are introduced here into the description. The sensor device 900 can be placed on a bed or directly on the floor. Therefore, the Z-axis represents the direction perpendicular to the ground, the direction away from the ground is the positive direction, the XY plane is parallel to the horizontal plane, and the X-axis is along the width direction of the sensor device 900. The Y-axis is along the length direction of the sensor device 900, and the origin O is located at the midpoint of an end edge of the sensor device 900. The YZ plane divides the sensor device 900 into left and right parts. Along the Y-axis direction, it can represent a relatively up and down direction. For example, the boundary between the back area and the waist area can be called the lower edge of the back area, and it is also the upper edge of the waist area.

The main body 901 can comprise an upper cover 911 and a lower cover 913. The upper cover 911 and the lower cover 913 enclose the first fiber-optic sensor 903 and the second fiber-optic sensor group 905 inside, and the upper cover 911 and the lower cover 913 are bonded together by stitches or adhesives. The main body 901 can be divided into a back area, a waist area, and a lower limb area along the Y-axis direction. The size of the main body 901 can be configured according to the body shapes and heights of the test subjects; for example, its length (along the Y-axis) can be 190 cm, and its width can be 85 cm, such size is suitable for most people, other suitable sizes can also be used, it is not limited here. Correspondingly, the width of the back area, waist area, and lower limb area (along the X-axis) of the main body 901 can also be configured according to the body shapes and heights of the test subjects. For example, the size suitable for most people is: 30 cm in the back area and 50 cm in the waist area of the main body; other suitable sizes of the main body can also be used, which is not limited here. When the subject lies supine on the sensor device 900 in a supine position, the back, waist, and lower limbs are placed in the back area, waist area, and lower limbs of the main body, and the upper limbs are placed in the back area and waist area of the main body. The upper cover 911 and the lower cover 913 can be made of various materials, such as leather or cotton.

The first fiber-optic sensor 903 is located in the back area. The first fiber-optic sensor 903 can be a fiber-optic sensor and can adopt a structure as shown in FIG. 5. In some embodiments, as shown in FIG. 9, the length (along the X-axis) of the first fiber-optic sensor 903 can be selected according to the test subject, for example, it can be 50 cm, and is suitable for most people; and the width (along the Y-axis) can be selected according to the test subject too, for example, it can be 30cm, and suitable for most people, or it can be other suitable sizes, which is not limited here. When the subject lies supine on the sensor device 900, the left and right body parts are roughly symmetrical along the Y-axis, the upper edge of the shoulder is aligned with the upper edge of the back area, the back is located in the back area of the main body, the legs are naturally brought close together, and the hands are naturally hanging down on both sides of the body, the subject's back is located on the first fiber-optic sensor 903 at this time. The first fiber-optic sensor 903 is used to acquire first vibration information of the subject.

The second fiber-optic sensor group 905 can include two or more fiber-optic sensors, and the two or more fiber-optic sensors (905-1, 905-2, . . . 905-n) can be sequentially arranged in the waist area along the Y-axis direction. The Y-axis direction can be the longitudinal axis direction of the main body, and the X-axis direction is the horizontal axis direction of the main body. Two or more fiber-optic sensors can adopt the structure of sensor device shown in FIG. 5. In some embodiments, as shown in FIG. 9, six fiber-optic sensors can be arranged in sequence along the Y-axis, and the width (along the Y-axis) of each fiber-optic sensor can be 1 cm-20 cm, and the length (along the X-axis) can be 10 cm-80 cm, other suitable size can also be used, it is not limited here. In other embodiments, the number of fiber-optic sensors in the second fiber-optic sensor group 905 can be changed; when the height of the test subject is particularly high, the number of fiber-optic sensors can be increased, for example, to eight or more sensors, so that the last fiber-optic sensor arranged in the Y-axis direction in the second fiber-optic sensor group 905 can be placed under the hip bone of the test subject when the test subject lies on its back. When the subject lies supine on the sensor device 900, his left and right body parts are roughly symmetrical along the Y-axis, the upper edge of his shoulder is aligned with the upper edge of the back area, his legs are naturally brought together, and hands are naturally hanging on both sides of the body; so that the waist and hip of the subject is located in the waist area of the sensor device. The second fiber-optic sensor group 905 is used to acquire second vibration information of the subject, and the second vibration information can include body vibration information detected by various sensors in the waist area of the sensor device.

The positioning indicator 907 is used for indicating and assisting the test subject to quickly lie on the preferred measuring position. As shown in FIG. 9, the positioning indicator 907 can be a shoulder stop, and the shoulder stop can be fixedly arranged on the upper cover 911 of the main body 901, for example, stitched to the upper cover 911. In some embodiments, the shoulder stop can also be detachably connected to the upper cover 911, for example, connected to the upper cover 911 by Velcro tape. In other embodiments, the positioning indicator 907 can include two or more shoulder stops, as shown in FIG. 10, illustrating a top view of three sensor devices, where the positioning indicator of the sensor device 1001 can include two shoulder stops 1011, being set on the side close to the demarcation line of the back area. A left shoulder stop and a right shoulder stop can be arranged on both sides of the Y-axis. The distance between the two stops is configured so that when the subject lies down, his neck is located between the left shoulder stop and the right shoulder stop, his left and right shoulders abut against the left shoulder stop and the right shoulder stop, respectively, and thus the shoulder of the subject is aligned with the upper edge of the back area. In some embodiments, the distance between the left shoulder stop and the right shoulder stop can be 130 mm. In some embodiments, the distance between the left shoulder stop and the right shoulder stop can be changed, and different distance can be selected according to subject of different body shapes. For example, when the shoulder stop is connected to the upper cover 911 by velcro, the size of the loop tap of the velcro on the upper cover 911 can be larger than the size of the hook tap on the shoulder stop, so that measurement assistants (such as medical staff) can adjust the position of the shoulder stop according to the body shape of the subject.

In some embodiments, the positioning indicator 907 can include one or more foot stops, for example, two foot stops, which are provided in the lower limb area of the sensor device for the feet or calves being pressed against when the subject is lying on the sensor device, so that the subject's legs are straightened and brought into a close position. In some embodiments, the foot stop can be fixedly arranged on the upper cover 911 of the main body 901, for example, connected to the upper cover 911 by stitching. In some embodiments, the foot stop can also be detachably connected to the upper cover 911, for example, connected to the upper cover 911 by velcro tape. In some embodiments, the distance between the left foot stop and the right foot stop can be 300 mm. As shown in FIG. 10, the sensor device 1001 can include two foot stops 1013. In some embodiments, the shape and color of the shoulder stop and the foot stop can be changed, and the present invention does not limit the shape and color thereof. For example, the positioning indicator of the sensor device 1003 shown in FIG. 10 includes two shoulder stops 1031 and two foot stops 1033.

In some embodiments, the positioning indicator 907 can include a neck pillow, which is arranged on the side close to the demarcation line of the back area, and placed in the center (near the Y-axis). The neck pillow can support the neck of the supine subject, so that the subject's shoulders are aligned with the upper edge of the back area. In some embodiments, the neck pillow can be fixedly arranged on the upper cover 911 of the main body 901, for example, connected to the upper cover 911 by stitching. In some embodiments, the neck pillow can also be detachably connected to the upper cover 911, for example, connected to the upper cover 911 by velcro tape. In some embodiments, the shape of the neck pillow can be cylindrical or approximately cylindrical to fit the curvature of the neck of the human body. As shown in FIG. 10, the positioning indicator 1051 of the sensor device 1005 is a neck pillow in an embodiment.

In some embodiments, the sensor device 900 can further include a support plate 909. The support plate 909 is used to support the first fiber-optic sensor 903 and the second fiber-optic sensor group 905, can be positioned under the first fiber-optic sensor 903 and the second fiber-optic sensor group 905, and is enclosed in the main body 901together with the first fiber-optic sensor 903 and the second fiber-optic sensor group 905. The support plate 909 can be a rigid structure, such as a wooden board, a PVC board, and the like.

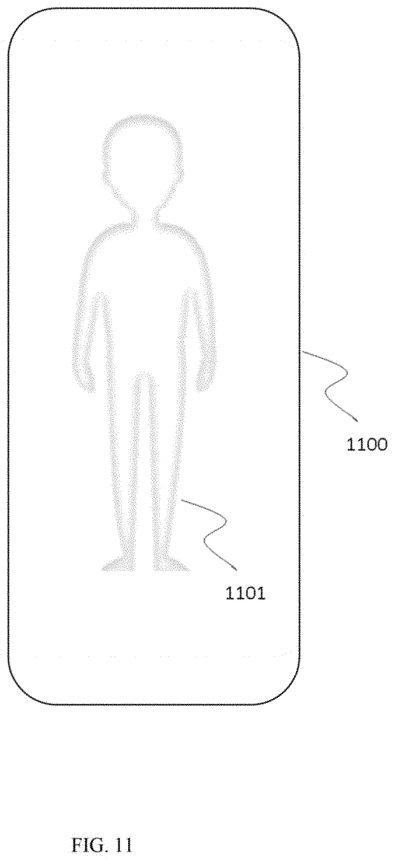

FIG. 11 is a schematic diagram of a positioning indicator in other embodiments of the present invention. In some embodiments, the upper cover 911 of the main body 901 in FIG. 9 can have three-dimensional structure. For example, as shown in FIG. 11, the upper cover of the sensor device 1100 can has a body-contour recess 1101. When the subject lies on the upper cover, the body rest on the body-contour recess 1101. The body-contour recess 1101 is arranged near the Y-axis of the sensor device and is symmetrically arranged along the Y-axis. When the subject lie on the body-contour recess 1101, the subject's head is located at the sensor device 1100, his back is located on back area of the sensor device 1100, his waist is located on the waist area of the sensor device 1100, and his lower limbs are located on the lower limb area of the sensor device 1100. In some embodiments, the sensor device can have different sizes according to the height of the subject. Correspondingly, body-contour recess 1101 can also change with the height and body shape of the subject. For example, if the size suitable for people with a height of 155cm-160cm is set to size S, the size suitable for people with a height of 161cm-170cm can be the S size adding a certain size, such as 2-5 cm. In some embodiments, the upper cover 911 of the main body 901 in FIG. 9 can be a planar structure, and then can set a contour line to identify the outline of the human body. For example, when the upper cover 911 is white, a red line can be used to identify the outline of the human body.

It should be noted that the above description is only a specific embodiment of this invention, and should not be regarded as the only embodiment. Obviously, for professionals in the field, after understanding the content and principles of the application, they can make various amendments and changes in form and details without departing from the principles and structure of the invention, but these amendments and changes are still within the protection scope of the claims of the present invention.

1. A method, comprising:
acquiring first vibration information of a supine subject from a first fiber-optic sensor by one or more processors, the first fiber-optic sensor being placed under a back section corresponding to the fourth thoracic vertebra of the supine subject;
acquiring second vibration information of the supine subject from a second fiber-optic sensor by the one or more processors, the second fiber-optic sensor being placed under a lumbar section corresponding to the fourth lumbar vertebra of the supine subject;
generating first hemodynamic related information on the basis of the first vibration information, and generating second hemodynamic related information on the basis of the second vibration information, by the one or more processors;
determining an aortic valve opening time of the supine subject on the basis of the first hemodynamic related information, and determining a pulse wave arrival time of the supine subject on the basis of the second hemodynamic related information, by the one or more processors; and
determining an aortic Pulse Wave Transit Time of the supine subject on the basis of the aortic valve opening time and the pulse wave arrival time by the one or more processors.

2. The method of claim 1, wherein the first fiber-optic sensor or the second fiber-optic sensor comprise:
an optical fiber, disposed substantially in a plane;
a light source, coupled with one end of the optical fiber;
a receiver, coupled to the other end of one optical fiber, and configured to sense changes in the intensity of light transmitted through the optical fiber; and
a mesh layer, composed of meshes with openings; the mesh layer is in contact with the surface of the optical fiber.

3. The method of claim 1, wherein the step of generating first hemodynamic related information on the basis of the first vibration information, and generating second hemodynamic related information on the basis of the second vibration information by one of more processors, further comprises step of:
filtering and scaling the first vibration information and the second vibration information to generate the first hemodynamic related information and the second hemodynamic related information.

4. The method of claim 1, wherein the step of determining an aortic valve opening time of the supine subject on the basis of the first hemodynamic related information by the one or more processors, further comprises steps of:
performing a second-order differential calculation on the first hemodynamic related information;
performing a feature search to a waveform of the first hemodynamic related information after the second-order differential calculation to determine the highest peak in a cardiac cycle; and
determining the aortic valve opening time of the supine subject based on the highest peak.

5. The method of claim 1, further comprising steps of:
acquiring a distance between the first fiber-optic sensor and the second fiber-optic sensor in a body height direction to generate an aortic pulse wave conduction distance by the one or more processors; and
determining an aortic Pulse Wave Velocity on the basis of the aortic pulse wave conduction distance and the aortic Pulse Wave Transit Time by the one or more processors.

6. The method of claim 5, further comprising step of:
sending at least one of the aortic Pulse Wave Transit Time and the aortic Pulse Wave Velocity to one or more output device, by the one or more processors.

7. A system, comprising:
a first fiber-optic sensor, being configured to be placed in an area corresponding to the fourth thoracic vertebra of a supine subject to acquire first vibration information of the supine subject;
a second fiber-optic sensor, being configured to be placed in an area corresponding to the fourth lumbar vertebra of the supine subject to acquire second vibration information of the supine subject;
one or more processors; and
one or more computer-readable storage medium having instructions stored thereon, which when being executed by the one or more processor, cause the one or more processors to perform steps of:
acquiring the first vibration information of the supine subject from the first fiber-optic sensor;
acquiring the second vibration information of the supine subject from the second fiber-optic sensor;
generating first hemodynamic related information on the basis of the first vibration information, and generating second hemodynamic related information on the basis of the second vibration information;
determining an aortic valve opening time of the supine subject on the basis of the first hemodynamic related information, and determining a pulse wave arrival time of the supine subject on the basis of the second hemodynamic related information; and
determining an aortic Pulse Wave Transit Time of the supine subject on basis of the aortic valve opening time and the pulse wave arrival time.

8. The system of claim 7, wherein the first fiber-optic sensor or the second fiber-optic sensor comprise:
an optical fiber, disposed substantially in a plane;
a light source, coupled with one end of one or more the optical fibers fiber;
a receiver, coupled to the other end of one optical fiber, and configured to sense changes in the intensity of light transmitted through the optical fiber; and
a mesh layer, composed of meshes with openings; the mesh layer is in contact with the surface of the optical fiber.

9. The system of claim 7, where the step of generating first hemodynamic related information on the basis of the first vibration information, and generating second hemodynamic related information on the basis of the second vibration information by one of more processors, further comprises step of:
filtering and scaling the first vibration information and the second vibration information to generate the first hemodynamic related information and the second hemodynamic related information.

10. The system of claim 7, where the step of determining an aortic valve opening time of the supine subject on the basis of the first hemodynamic related information, further comprises steps of:
performing a second-order differential calculation on the first hemodynamic related information;
performing a feature search to a waveform of the first hemodynamic related information after the second-order differential calculation to determine the highest peak in a cardiac cycle; and
determining the aortic valve opening time of the supine subject based on the highest peak.

11. The system of claim 7, where the one or more processors are configured to execute steps of:
acquiring a distance between the first fiber-optic sensor and the second fiber-optic sensor in a body height direction to generate an aortic pulse wave conduction distance; and
determining an aortic Pulse Wave Velocity on the basis of the aortic pulse wave conduction distance and the aortic Pulse Wave Transit Time.

12. The system of claim 11, wherein the one or more processors are configured to execute step of:
sending at least one of the aortic Pulse Wave Transit Time and the aortic Pulse Wave Velocity to one or more output device.

13. A device, comprising:
a main body, used for a subject to lie down, comprising an upper cover and a lower cover, and having a back area and a waist area;
a first fiber-optic sensor, being placed in the back area of the main body and used for acquiring first vibration information of the supine subject; and
a second fiber-optic sensor group, comprising two or more fiber-optic sensors, being placed in the waist area of main body and used for acquiring second vibration information of the supine subject;
wherein the upper cover and lower cover together enclose the first fiber-optic sensor and the second fiber-optic sensor group therein.

14. The device of claim 13, wherein the device comprises a neck pillow; the neck pillow is set on the upper cover for supporting the neck of the supine subject whereby the subject can be located on the measuring position.

15. The device of claim 13, wherein the device comprises shoulder stops; the shoulder stops are set on the upper cover for the shoulder of the supine subject to abut against whereby the supine subject can be located on the measuring position.

16. The device of claim 13, wherein the main body comprises a lower limb area; the device comprises foot stops; the foot stops are set on the lower limb area of the upper cover for the feet or calves of the supine subject to abut against whereby the supine subject can be located on the measuring position.

17. The device of claim 13, wherein the upper cover of the main body is configured as a three-dimensional structure, and defines a body-contour recess whereby the supine subject can be located on the measuring position.

18. The device of claim 13, wherein two or more fiber-optic sensors of the second fiber-optic sensor group are configured to arrange along the longitudinal axis of the main body.

19. The device of claim 13, wherein the fiber-optic sensor comprises:
an optical fiber, disposed substantially in a plane;
a light source, coupled with one end of the optical fiber;
a receiver, coupled to the other end of one optical fiber, and configured to sense changes in the intensity of light transmitted through the optical fiber; and
a mesh layer, composed of meshes with openings; the mesh layer is in contact with the surface of the optical fiber.
